(19) United States
(12) Patent Application Publication
(10) Pub. No.: US 2022/0169990 A1
(43) Pub. Date: Jun. 2, 2022

(54) METHOD FOR PRODUCING CARDIOMYOCYTE

(71) Applicants: I Peace, Inc., Palo Alto, CA (US); ID Pharma Co., Ltd., Tokyo (JP)

(72) Inventors: Koji TANABE, Palo Alto, CA (US); Kenta SUTO, Palo Alto, CA (US); Tsugumine SHU, Tokyo (JP); Toyotaka MORI, Tokyo (JP)

(73) Assignees: I Peace, Inc., Palo Alto, CA (US); ID Pharma Co., Ltd., Tokyo (JP)

(21) Appl. No.: 17/602,428

(22) PCT Filed: Mar. 31, 2020
(86) PCT No.: PCT/JP2020/014733

§ 371 Date: Oct. 8, 2021

Related U.S. Application Data
(60) Provisional application No. 62/831,573, filed on Apr. 9, 2019.

(51) Int. Cl. C12N 5/077; C12N 15/86; C12N 7/00

(52) U.S. Cl. CPC C12N 5/0657; C12N 15/86; C12N 2760/18821; C12N 2506/45; C12N 2760/18841; C12N 7/00

(57) ABSTRACT

A method for producing a cardiomyocyte including preparing a stem cell, introducing a Sendai virus into the stem cell by infection, expressing mRNA for synthesizing an inducing factor from the Sendai viruses in the stem cell to induce a cardiomyocyte from the stem cell.

### TECHNICAL FIELD

The present invention relates to a cell technique, which is a method for producing a cardiomyocyte.

### BACKGROUND ART

Induced pluripotent stem cells (iPS cells) can be transformed into any of the cells that form the body. Therefore, iPS cells, which can be transformed into various types of somatic cells and tissues, are expected to be used for cell transplantation therapy and drug discovery research. In addition, for example, in 2014, retinal cells produced from iPS cells were applied in transplantation therapy. Not only in Japan, but also in other countries throughout the world, projects are underway to produce brain cells and various organ cells from iPS cells and use them for transplantation therapy.

In the prior art, there are many methods for transforming iPS cells into differentiated cells. However, in order to use iPS cells for transplantation therapy, it is important to establish an efficient method for inducing iPS cells. Specifically, it is necessary to establish a technology to be used when iPS cells are induced into differentiated cells, in which the efficiency and accuracy of induction can be improved, and in which the functionality of the produced differentiated cells can suit transplantation therapy.

In the prior art, a method for inducing iPS cells or embryonic stem cells (ES cells) into differentiated cells has been performed by imitating development processes by combining hormones and growth factors that determine cell properties with a low-molecular-weight compound, and changing their amount ratios and concentrations over time. However, it is difficult and inefficient to completely imitate these development processes in vitro. In addition, in the case of humans, a much longer induction period is required compared to an induction period of mouse somatic cells. For example, it takes three months or longer in order to produce mature differentiated cells in the prior art.

In addition, the efficiency of induction varies greatly depending on ES/iPS cell lines, and there are problems such as non-uniform properties of the induced somatic cells. Actually, it was indicated that, when chemical substances were added to a plurality of ES cell clones to produce various cells, there were clones that were easily induced into pancreatic cells and clones that were easily induced into cardiac cells, and there were differences in ease of induction depending on clones (for example, refer to Non-Patent Document 1). In addition, when nerve cells were produced from dozens of types of iPS cells using a method in which iPS cells were cultured in a culture medium containing no serum or chemical substance that inhibits induction into nerve cells, which is called a serum-free floating culture of embryoid body-like aggregates with quick reaggregation (SFEBq method), to produce nerve cells from iPS cells/ES cells, it was proved that there were iPS/ES cell clones that were unlikely to be transformed into nerve cells (for example, refer to Non-Patent Document 2).

Specifically, it has been confirmed that cells induced from human ES/iPS cells using a method utilising hormones or chemical substances are fetal somatic cells in the early stage. Induction of mature human somatic cells is very difficult, and requires a long-term culture over several months. However, in drug discovery and transplantation medicine targeting individuals who have completed development, it is important to produce somatic cells that match the maturity of the individual.

On the other hand, a method in which genes that define properties of specific somatic cells are directly introduced into ES/iPS cells using deoxyribonucleic acid (DNA) viruses that become integrated into genomes and desired somatic cells are produced has been proposed. However, in the method in which the stem cells are induced into the somatic cells using the DNA viruses that become integrated into the genomes in order to express the specific genes, the genes are inserted into the genomes of ES/iPS cells and damage endogenous genes. As a result, there is a problem that drug discovery screening may not be performed correctly and there is a risk of canceration in transplantation (for example, refer to Non-Patent Documents 3 and 4).

### PRIOR ART DOCUMENT

### Non-Patent Document

Non-Patent Document 1: Nature Biotechnol 26(3): 313-315, 2008.
Non-Patent Document 2: PNAS, 111: 12426-12431, 2014
Non-Patent Document 3: N Eng J Med, 346: 1185-1193, 2002
Non-Patent Document 4: Science 302: 415-419, 2003

### SUMMARY OF INVENTION

### Problem to be Solved by the Present Invention

An object of the present invention is to provide a method for producing a cardiomyocyte which makes it possible to efficiently produce cardiomyocyte in a short period without damaging cell genes.

### Solution to Solve the Problem

According to an aspect of the present invention, a method for producing a cardiomyocyte including preparing a stem cell, introducing a Sendai virus into the stem cell by infection, expressing mRNA for synthesizing an inducing factor from the Sendai virus in the stem cell to induce a cardiomyocyte from the stem cell is provided.

In the above method for producing the cardiomyocyte, the stem cell may be an induced pluripotent stem cell.

In the above method for producing the cardiomyocyte, the stem cell may be an embryonic stem cell.

In the above method for producing the cardiomyocyte, the inducing factor may include at least one selected from the group consisting of GATA, Myocyte-specific enhancer factor (MEF), and TBX.

In the above method for producing the cardiomyocyte, the cardiomyocyte may be at least one selected from the group consisting of a pacemaker cell, a cardiomyocyte, a smooth muscle cell, and an endothelial cell.

In the above method for producing the cardiomyocyte, at least one marker selected from the group consisting of cTnT, MYH6, MYH7, MYL2, MYL7, TNNT2, NKX2.5, TBX5, SIRPA, miR1, mi208a, and mi499a-p5 may be positive in the cardiomyocyte.

In the above method for producing the cardiomyocyte, the Sendai virus may express mRNA of a drug-resistant gene in the stem cell.

In the above method for producing the cardiomyocyte, the drug-resistant gene may be at least one selected from the group consisting of a puromycin-resistant gene, a blasticidin-resistant gene, a hygromycin-resistant gene, and a neomycin-resistant gene.

The above method for producing the cardiomyocyte may include selecting a cell exhibiting drug resistance after the stem cell is infected with the Sendai virus.

In the above method for producing the cardiomyocyte, in the induced cardiomyocyte, the Sendai virus may not have to be integrated into a genome.

### BRIEF DESCRIPTION OF DRAWINGS

FIG. 1 shows microscope images of cells according to an example.

FIG. 2 shows microscope images of cell colonies according to an example.

FIG. 3 shows a fluorescence microscope image of cell colonies according to an example.

FIG. 4 is a graph showing the number of beating cell colonies according to an example.

FIG. 5 shows histograms of results of cell analysis by flow cytometry according to an example.

FIG. 6 is an image showing analysis results of gene expression according to an example.

FIG. 7 is an image showing analysis results of gene expression according to an example.

FIG. 8 is an image showing analysis results of gene expression according to an example.

### DESCRIPTION OF EMBODIMENTS

Hereinafter, embodiments of the present invention will be described in detail. Here, the following embodiments exemplify a device and a method for embodying the technical ideas of the invention. The technical ideas of the invention do not specify combinations of constituting members and the like as the following. The technical ideas of the invention can be variously modified within the scope of the claims.

A method for producing somatic cells from stem cells according to an embodiment of the present invention includes preparing stem cells and introducing Sendai viruses into the stem cells by infection, expressing mRNA for synthesizing an inducing factor from the Sendai viruses in the stem cells to induce cardiomyocytes from the stem cells. In the present method, Sendai viruses may be introduced into stem cells in vitro by infection.

As the stem cells, both induced pluripotent stem cells (iPS cells) and embryonic stem cells (ES cells) can be used. The stem cells may be human stem cells or non-human animal stem cells.

Induction refers to reprogramming, initialization, transformation, cell transdifferentiation (transdifferentiation or lineage reprogramming), induced differentiation, cell fate change (cell fate reprogramming) and the like.

Examples of cardiomyocytes to be induced include pacemaker cells, cardiomyocytes, smooth muscle cells, and endothelial cells.

Sendai viruses (SeV) introduced into stem cells have RNA genomes that express mRNA for synthesizing an inducing factor for inducing stem cells into cardiomyocytes. Here, production of recombinant Sendai virus vectors that can express arbitrary mRNA is outsourced, for example, using the patented technique (held by ID Pharma Co., Ltd.). Examples of mRNA for synthesizing an inducing factor, which is mRNA expressed by RNA genomes of Sendai viruses, include mRNA of GATA genes, mRNA of MEF genes, mRNA of TBX genes, mRNA of MYOCD genes, mRNA of MESP genes, and miR-133. Examples of GATA genes include GATA4A genes. Examples of MEF genes include MEF2C genes. Examples of TBX genes include TBX5 genes. Examples of MESP genes include MESP1 genes.

mRNA for synthesizing an inducing factor, which is mRNA expressed by RNA genomes of Sendai viruses, may be used singly. Alternatively, mRNA of GATA genes, mRNA of MEF genes, and mRNA of TBX genes may be combined. mRNA of GATA genes, mRNA of MEF genes, mRNA of TBX genes, mRNA of MYOCD genes, and mRNA of MESP genes may be combined. mRNA of GATA genes, mRNA of MEF genes, mRNA of TBX genes, mRNA of MYOCD genes, mRNA of MESP genes, and miR-133 may be combined.

For example, Sendai viruses introduced into stem cells express at least one selected from among mRNA of GATA genes, mRNA of MEF genes, and mRNA of TBX genes as mRNA for synthesizing an inducing factor.

Sendai viruses may have RNA genomes that express mRNA of drug-resistant genes. Drugs are antibiotics, for example, puromycin, blasticidin, hygromycin, neomycin, G418, and Zeocin. For example, Sendai viruses introduced into stem cells express mRNA of drug-resistant genes. Cells in which mRNA of drug-resistant genes is expressed exhibit drug resistance.

When Sendai viruses express mRNA of drug-resistant genes, cells exhibiting drug resistance may be selected after the infection. For example, if Sendai viruses express at least one mRNA of puromycin-resistant genes, blasticidin-resistant genes, hygromycin-resistant genes, neomycin-resistant genes, G418-resistant genes, and Zeocin (registered trademark)-resistant genes, when cells after infection are exposed to a corresponding antibiotic, it is possible to kill cells other than those into which Sendai viruses are introduced and it is possible to select cells into which Sendai viruses are introduced.

Sendai viruses may have RNA that can express, for example, mRNA including mRNA of GATA genes, mRNA of MEF genes, mRNA of TBX genes, and mRNA of puromycin-resistant genes (hereinafter referred to as “GATA-MEF-TBX-Puro mRNA”). Cells in which GATA-MEF-TBX-Puro mRNA is expressed produce GATA, MEF, and TBX and exhibit puromycin resistance.

Sendai viruses may have RNA that can express, for example, mRNA including mRNA of GATA4A genes, mRNA of MEF2C genes, mRNA of TBX5 genes, and mRNA of puromycin-resistant genes (hereinafter referred to as “GATA4A-MEF2C-TBX5-Puro mRNA”). Cells in which GATA4A-MEF2C-TBX5-Puro mRNA is expressed produce GATA4A, MEF2C, and TBX5 and exhibit puromycin resistance.

Stem cells are cultured on a substrate coated with Matrigel, laminin, vitronectin or the like. Sendai viruses are introduced into stem cells by being suspended in a cell culture solution. Sendai viruses recognize cell surface antigens and infect stem cells.

The density of stem cells during infection with Sendai viruses is, for example, 0.2×10⁴ cells/well to 1.0×10⁶ cells/well, 1.0×10⁴ cells/well to 2.0×10⁵ cells/well, or 1.0×10⁵ cells/well to 5.0×10⁵ cells/well in a 12-well plate well.

The titer of Sendai viruses used is, for example, 1×10¹² CIU/mL to 1×10⁵ CIU/mL, 1×10¹⁰ CIU/mL to 1×10⁶ CIU/mL, or 1×10⁹ CIU/mL to 1×10⁷ CIU/mL. The multiplicity of infection (MOI) of Sendai viruses is, for example, 0.1 to 100.0, 1.0 to 50.0, or 1.0 to 20.0 at a time.

The culture medium used for culturing stem cells is, for example, a stem cell culture medium such as mTeSR1, TeSR2, (registered trademark, Stemcell Technologies Inc.), and Stem Fit (commercially available from Reprocell Inc.). The culture medium may contain a ROCK (Rho-associated coiled-coil forming kinase/Rho binding kinase) inhibitor.

The culture medium used for infection with Sendai viruses may contain, for example, activin, bone morphogenetic protein 4 (BMP4), basic fibroblast growth factor (bFGF), vascular endothelial growth factor (VEGF), and IWP-2. Examples of culture mediums used for infection with Sendai viruses include Cardiomyocyte Differentiation Medium A (Gibco). Examples of culture mediums used for culturing cells infected with Sendai viruses include Cardiomyocyte Differentiation Medium B (Gibco).

The culture medium used during and before and after infection with Sendai viruses may contain B18R proteins. The B18R proteins alleviate the congenital antiviral response of cells. The B18R proteins may be used to suppress cell death due to an immune response associated with virus infection. However, in the method according to the embodiment, since cardiomyocytes can be produced from stem cells in a short period, the culture medium does not have to contain B18R proteins or may contain B18R proteins in a dilute concentration such as 0.01% to 1%.

Stem cells are induced into cardiomyocytes within 25 days, 20 days, 10 days, 9 days, 8 days, 7 days, 6 days, 5 days or 4 days after infection with Sendai viruses. Whether stem cells have been induced into cardiomyocytes is confirmed according to whether at least one selected from among, for example, cTnT, MYH6, MYH7, MYL2, MYL7, TNNT2, NKX2.5, TBX5, SIRPA, miR1, miR208a, and miR499a-p5, is positive.

According to the method for the embodiment of the present invention described above, by having stem cells express mRNA for synthesizing an inducing factor, it is possible to efficiently produce cardiomyocytes without damaging genes of the stem cells and without integration.

In the method for producing cardiomyocytes from stem cells using only hormones and chemical substances, a very long period is required until cardiomyocytes are produced. However, according to the method for the embodiment of the present invention, it is possible to produce cardiomyocytes in a short period and with high efficiency.

In addition, in the method for producing cardiomyocytes from stem cells using only hormones and chemical substances, only some of the stem cells are transformed into desired cardiomyocytes. However, according to the method for the embodiment of the present invention, most of the cells that have been infected with Sendai viruses can become desired cardiomyocytes.

In addition, in the method for producing cardiomyocytes from stem cells using only hormones and chemical substances, even if the same protocol is used, there is a variation among clones with clones that become desired cardiomyocytes and clones that do not become desired cardiomyocytes. However, according to the method for the embodiment of the present invention, it is possible to obtain a high induction efficiency with a plurality of clones.

In addition, when cells for transplantation are produced by induction from an undifferentiated cell population using cytokines or the like, cells that have not been induced may remain in the cells for transplantation. There is a risk that these remaining uninduced cells will independently divide and proliferate at the transplantation site and form teratomas and the like. However, according to the method for the embodiment of the present invention, since mRNA of drug-resistant genes can be expressed at the same time, it is possible to select cells into which Sendai viruses are introduced by using a drug. Therefore, it is possible to avoid risks such as contamination of uninduced cells and formation of teratomas, which is suitable for transplantation medicine.

In addition, according to the method for the embodiment of the present invention, Sendai viruses are used, and viruses that insert genes into genomes are not used. Therefore, without integration, genes of stem cells are not damaged, and since there is no risk of canceration of the produced cardiomyocytes, the method can be clinically used.

Furthermore, for example, if iPS cells are produced from blood cells in a clean environment of a completely closed system, and consecutively, according to the method for the embodiment of the present invention, cardiomyocytes are produced from iPS cells in a clean environment of a completely closed system, it is possible to produce cleaner and safer cardiomyocytes.

In addition, according to the method for the embodiment of the present invention, since cardiomyocytes can be produced in a short period, it is not necessary to use B18R or the like, or even if it is used, its concentration can be very low.

### EXAMPLE

Sendai viruses that can express GATA4A-MEF2C-TBX5-Puro mRNA were prepared by ID Pharma Co., Ltd. The titer of the prepared Sendai viruses was 22×10⁸ CIU/mL.

A 12-well dish coated with a solubilized basement membrane preparation (Matrigel, Corning) was prepared, and a feeder-free culture medium (mTeSR (registered trademark) 1, Stemcell Technologies) containing a ROCK (Rho-associated coiled-coil forming kinase/Rho binding kinase) inhibitor (Selleck) at a concentration of 10 nmol/mL was put into each well. The ROCK inhibitor suppresses cell death.

iPS cells were dispersed in a tissue-cultured cell detachment/separation/dispersion solution (Accutase, Innovative Cell Technologies) and seeded in a 12-well dish. iPS cells to be infected with Sendai viruses were seeded at a density of 2×10⁵ cells per well. Control iPS cells that were not to be infected with Sendai viruses were seeded at a density of 2.0×10⁵ cells per well. Then, the iPS cells were cultured in a feeder-free culture medium under gas conditions of 5% carbon dioxide concentration and 20% oxygen concentration for 24 hours.

The culture medium was replaced with a Cardiomyocyte Differentiation Medium A (Gibco), and iPS cells were infected with Sendai viruses that can express GATA4A-MEF2C-TBX5-Puro mRNA so that the MOI was 10 or 20. Some of the iPS cells were not infected with Sendai viruses as a control.

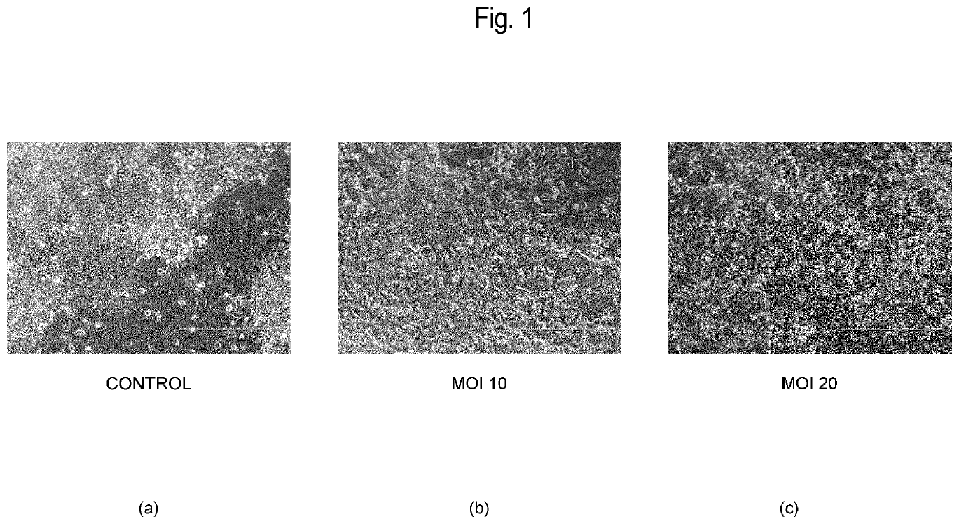

As shown in FIG. 1, on the second day after the infection with the Sendai viruses, the morphology of cells infected with the Sendai viruses was different from the morphology of cells not infected with the Sendai viruses, and indicated the morphology of differentiated cells. Specifically, the cells infected with the Sendai viruses were larger in size and had an increased proportion of cytoplasm.

On the second day after the infection with the Sendai viruses, the culture medium in the wells was replaced with Cardiomyocyte Differentiation Medium B (Gibco). On the 4th day after the infection with the Sendai viruses, the culture medium in the wells was replaced with Cardiomyocyte Maintenance Medium (Gibco). Then, the Cardiomyocyte Maintenance Medium (Gibco) was replaced every two days.

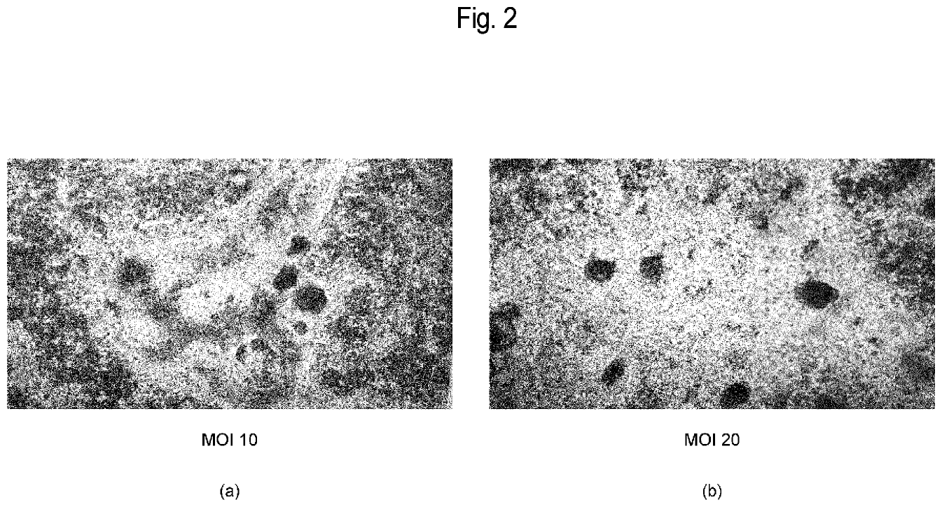

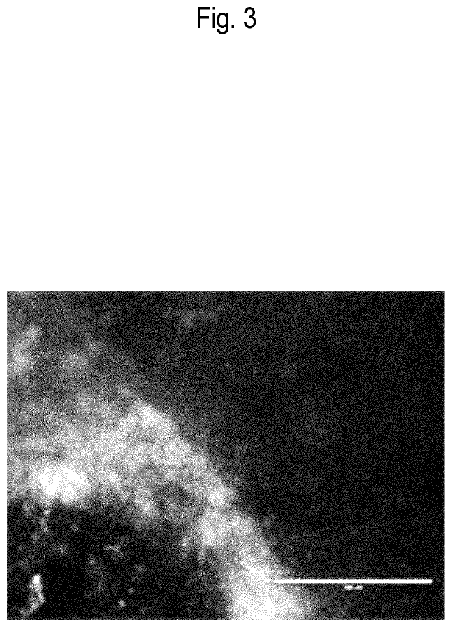

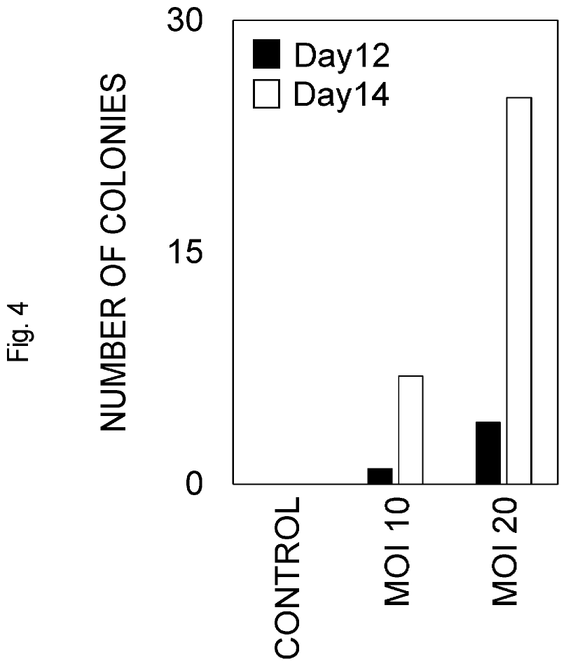

As shown in FIG. 2, from the 7th day to 12th day after the infection with the Sendai viruses, it was confirmed that the cells formed myocardial tissues and the myocardial tissues were beating. When beating tissues were stained with fluorescently labeled anti-troponin T antibodies, the beating tissues were TnT positive as shown in FIG. 3. In addition, on the 12th day and 14th day after the infection with the Sendai viruses, as shown in FIG. 4, a plurality of colonies composed of cells infected with the Sendai viruses were beating, but cells not infected with the Sendai viruses did not form beating colonies.

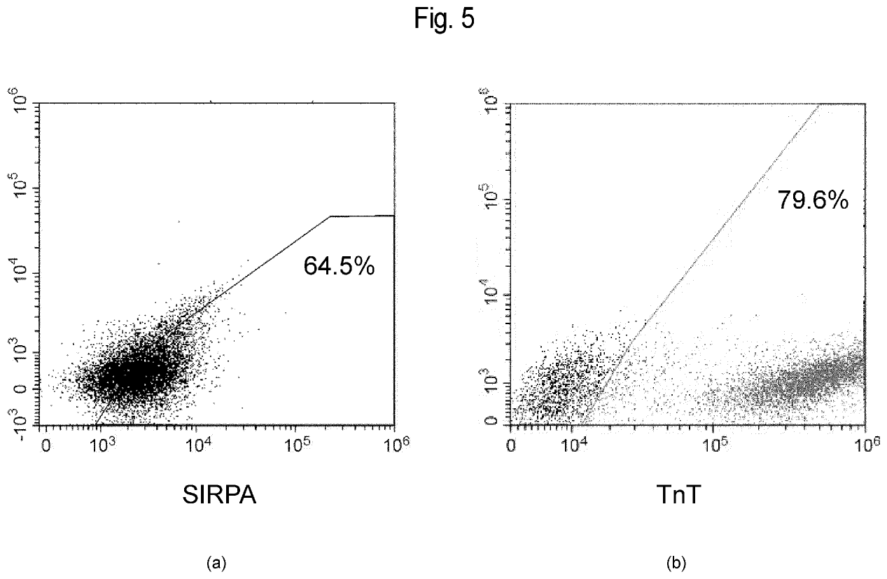

On the 12th day after the infection with the Sendai viruses, cells infected with the Sendai viruses at an MOI of 10 were analyzed with a flow cytometer, and as shown in FIG. 5, the signal regulatory protein α (SIPRA) serving as a myocardial marker was positive in 64.5% of the cells, and troponin (TnT) was positive in 79.6% of the cells.

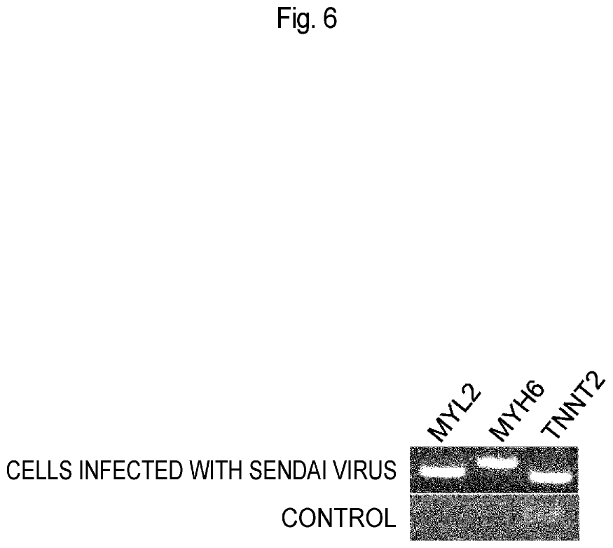

On the 12th day after the infection with the Sendai viruses, gene expression in cells infected with the Sendai viruses and gene expression in control cells were analyzed through semi-quantitative PCR, as shown in FIG. 6. It was confirmed that myocardial markers MYL2, MYH6, and TNNT2 were expressed in the cells infected with the Sendai viruses.

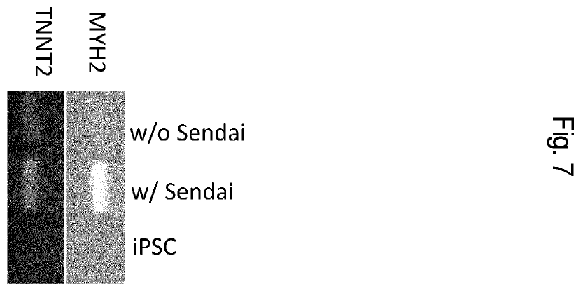

On the 12th day after the infection with the Sendai viruses, gene expression in cells infected with the Sendai viruses, gene expression in control cells, and gene expression in iPS cells not cultured in the Cardiomyocyte Differentiation Medium B (Gibco) were analyzed through semi-quantitative PCR, as shown in FIG. 7. It was confirmed that myocardial markers MYH2 and TNNT2 were expressed in cells infected with Sendai viruses.

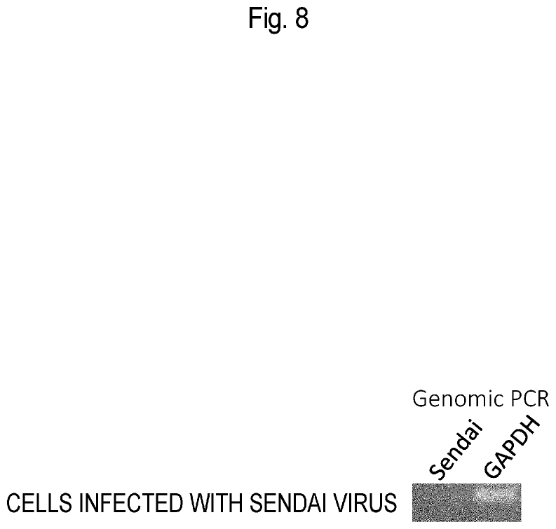

On the 12th day after the infection with the Sendai viruses, gene expression in cells infected with the Sendai viruses was analyzed through semi-quantitative PCR, as shown in FIG. 8. No expression of nuclear protein (NP) genes of Sendai viruses was observed and it was confirmed that Sendai viruses were not integrated into the genome.

1. A method for producing a cardiomyocyte, comprising:
preparing a stem cell; and
introducing a Sendai virus into the stem cell by infection, expressing mRNA for synthesizing an inducing factor from the Sendai virus in the stem cell to induce cardiomyocyte from the stem cell,
wherein the inducing factor includes GATA, Myocyte-specific enhancer factor (MEF), and TBX, and
wherein the multiplicity of infection of the Sendai virus is 0.1 to 100.0 per one time.

2. The method for producing the cardiomyocyte according to claim 1,
wherein the stem cell is an induced pluripotent stem cell.

3. The method for producing the cardiomyocyte according to claim 1,
wherein the stem cell is an embryonic stem cell.

4. (canceled)

5. The method for producing the cardiomyocyte according to claim 1,
wherein the cardiomyocyte is at least one selected from the group consisting of a pacemaker cell, a cardiomyocyte, a smooth muscle cell, and an endothelial cell.

6. The method for producing the cardiomyocyte according to claim 1,
wherein, at least one marker selected from the group consisting of cTnT, MYH6, MYH7, MYL2, MYL7, TNNT2, NKX2.5, TBX5, SIRPA, miR1, mi208a, and mi499a-p5 is positive in the cardiomyocyte.

7. The method for producing the cardiomyocyte according to claim 1,
wherein the Sendai virus expresses mRNA of drug-resistant gene in the stem cell.

8. The method for producing the cardiomyocyte according to claim 7,
wherein the drug-resistant gene is at least one selected from the group consisting of a puromycin-resistant gene, a blasticidin-resistant gene, a hygromycin-resistant gene, and a neomycin-resistant gene.

9. The method for producing the cardiomyocyte according to claim 7, comprising
selecting a cell exhibiting drug resistance after the stem cell is infected with the Sendai virus.

10. The method for producing the cardiomyocyte according to claim 1,
wherein, in the induced cardiomyocyte, the Sendai virus is not integrated into a genome.

11. The method for producing the cardiomyocyte according to claim 1,
wherein the multiplicity of infection of the Sendai virus is 1.0 to 50.0 per one time.

12. The method for producing the cardiomyocyte according to claim 1,
wherein the multiplicity of infection of the Sendai virus is 1.0 to 20.0 per one time.
